(19) United States
(12) Patent Application Publication
(10) Pub. No.: US 2009/0117631 A1
(43) Pub. Date: May 7, 2009

(54) ALCOHOL EXTRACTION PROCESS FOR BIOFUEL PRODUCTION

(76) Inventors: Pierre Lucien Cote, Dundas (CA); Steven Kristian Pedersen, Quebec (CA)

Correspondence Address: BERESKIN AND PARR, 40 KING STREET WEST, BOX 401, TORONTO, ON, M5H 3Y2, CA

(21) Appl. No.: 12/259,788

(22) Filed: Oct. 28, 2008

Related U.S. Application Data
(60) Provisional application No. 60/984,936, filed on Nov. 2, 2007.
(60) Provisional application No. 60/984,923, filed on Nov. 2, 2007.

(51) Int. Cl. C12M 1/12; C12P 7/06

(52) U.S. Cl. 435/161; 435/289.1; 210/640; 210/180

(57) ABSTRACT

This document describes a fermentation product producing or processing apparatus or process involving membrane pervaporation (PV) and either vapor permeation or distillation or both. The fermentation product may be produced semi-continuously wherein product concentration is maintained below a selected value by removal through pervaporation membranes. After a period of operation, the broth may be distilled. Distillation and/or pervaporation products may be further dewatered using vapor permeation. The PV membranes may be used in the form of immersed modules, for example with a flat sheet configuration.

This application claims the benefit of U.S. patent application No. 60/984,936 and 60/984,923 both filed on Nov. 2, 2007 both of which are incorporated herein in their entirety by this reference to them.

### FIELD

This specification relates to processing or producing fermentation products, for example acetone, isopropanol, butanol, ethanol or mixtures such as ABE or IBE, involving membrane pervaporation.

### BACKGROUND

The following is not an admission that anything discussed herein is prior art or common knowledge of persons skilled in the art.

An ethanol processing process involving membrane pervaporation was proposed in FIG. 9 of A Review of Pervaporation for Product Recovery from Biomass Fermentation Processes (Vane 2005). The proposed process involved pumping a 5 wt % ethanol fermentation product from a feed tank to a hydrophobic pervaporation module to produce a permeate vapor of about 20 wt % ethanol. This vapor would be fed to a fractional condenser to produce an overhead product of about 95 wt % ethanol. This overhead would then be condensed and pumped to a hydrophilic pervaporation module to produce a retentate product of about 99.5 wt % ethanol.

U.S. patent application Ser. No. 11/494,900 by Mairal et al., published as US 2007/0031954 A1 on Feb. 8, 2007, describes a process for recovering light alcohols using a combination of steps including fermentation, first membrane separation, dephlegmation and dehydration by second membrane separation. U.S. Pat. No. 6,755,975 describes related technology. These publications also summarize background information or available alternatives regarding these processes. Publication US 2007/0031954 A1 is incorporated herein in its entirety by this reference to it.

U.S. Pat. No. 5,755,967 describes the use of pervaporation in butanol extraction and is incorporated herein in its entirety by this reference to it.

### SUMMARY

The following summary is intended to introduce the reader to the invention and not to limit or define any claimed invention.

This document describes a fermentation product processing apparatus or process involving membrane pervaporation (PV) and either vapor permeation or distillation or both. The fermentation produced may be produced semi-continuously. The product may be, for example, acetone, isopropanol, butanol or ethanol or mixtures of them (ABE or IBE). Distillation, if any, may be performed after pervaporation.

### BRIEF DESCRIPTION OF THE DRAWINGS

FIG. 1 shows a process flow diagram for an ethanol production plant.

FIG. 2 shows two alternate permeate handling systems for the plant of FIG. 1.

FIG. 3 shows a cross-section of a membrane element.

FIG. 4 shows a side view of the membrane element of FIG. 3.

FIG. 5 shows a cassette of membrane elements of FIG. 3.

FIGS. 6 and 7 are sections of process flow diagrams corresponding to examples.

### DETAILED DESCRIPTION

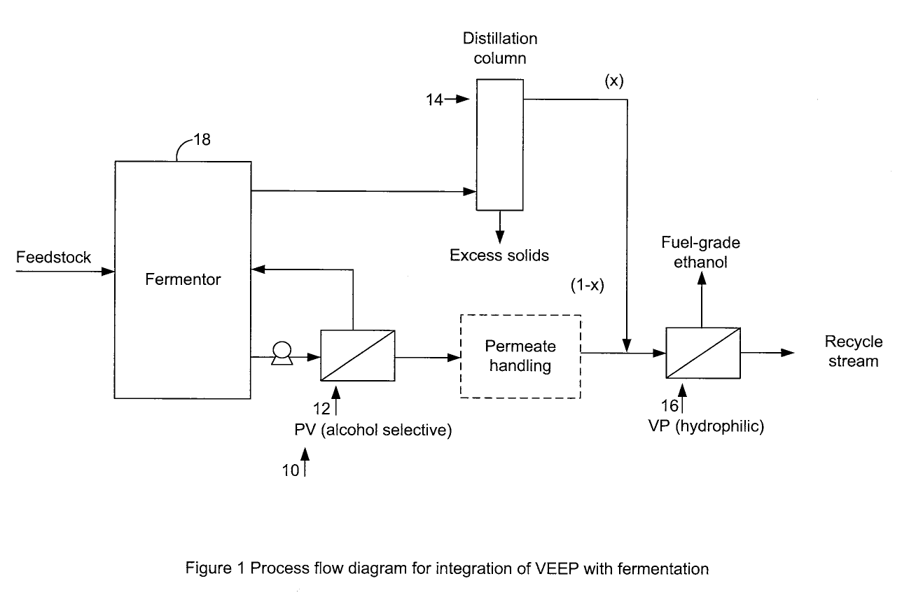

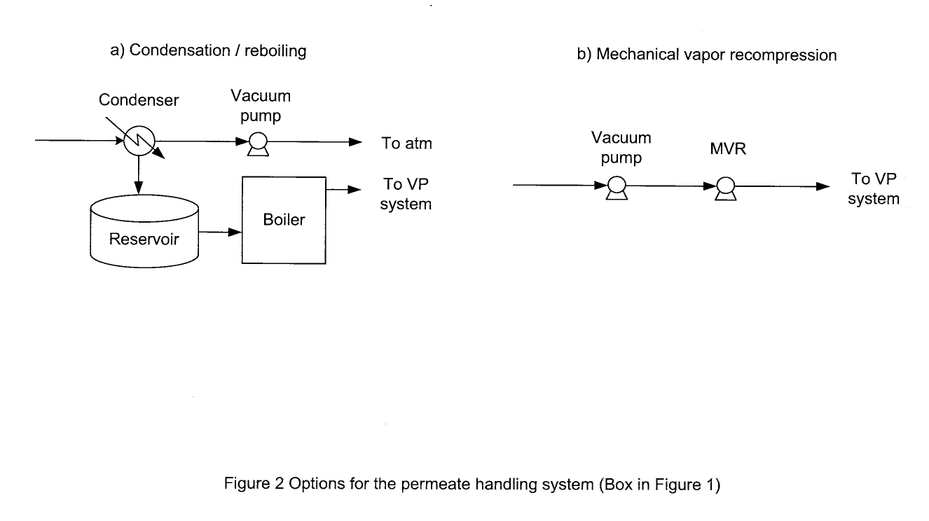

Referring to FIG. 1, a fermentation product production plant 10 has a product extraction pervaporation system 12, a distillation column 14, a vapour-permeation dewatering system 16 and a fermentor 18.

The fermentor 18 may be operated continuously, but is preferably operated semi-continuously. Under continuous operation, the fermentor is operated in steady-state, with continuous feedstock addition, continuous parallel flows to the pervaporation system 12 and distillation column 14, for extended periods of operation. Under semi-continuous operation, the fermentor is operated in an extended batch mode, with some feedstock added at a time and rate to generally correspond to product removed through the pervaporation system 12 during the batch.

Under semi-continuous operation, the pervaporation system 12 may be operated only during a portion of the time that the fermentator is operated. For example, the pervaporation system 12 may start to be operated only after the product content in the fermentation broth reaches a level that materially inhibits fermentation, for example 50% of a near-fully inhibitory level. Once started, the pervaporation system 12 is operated to remove product so that the product concentration in the broth is kept below a selected level, for example a level at which product inhibition is significant. For example, with ethanol production using a feed of sugars or starches (corn, sugar-cane) the selected level may be 2-7 wt % since the rate of ethanol production declines noticeably when ethanol concentration in the broth exceeds about 2 wt % and is reduced to negligible values when ethanol concentration reaches about 5-14 wt %. For butanol processing, product inhibition may become noticeable at butanol concentrations of 0.5 wt % and the selected level may be at about that level. As future work on yeasts and enzymes hopefully increases the product tolerance of all of these systems, the selected level may be raised upwards since a higher selected level increases the efficiency of product pervaporation.

In a semi-continuous or extended batch process, some inhibitory compounds, such as salts, glycerol or organic acids, built up in the broth over time. When the concentrations of one or more selected non-product inhibitory compounds reaches a selected level, for example a level that materially inhibits product alcohol production, or when the rate of fermentation product production declines to below a selected rate, the batch is complete. Pervaporation may continue to remove remaining product from the broth, or the broth may be sent from the fermentor 18 to the distillation column 14, or both processes may work on the broth simultaneously. In one example, until the end of the batch there is no flow of broth from the fermentor 18 to the distillation column 14. Alternately, there may be a flow of broth in parallel to the distillation column 14 and the pervaporation system 12 during all or part of the batch.

An example of an operation cycle includes the following steps. In a first step, an empty fermentor 18 is loaded up for a batch, this task taking time duration T0. In the next step, fermentation occurs within a closed fermentor 18 for duration T1 until the product alcohol reaches a selected concentration in the broth, for example about half of the known product inhibition level. In the next step, fermentation continues but the broth, or a portion of the broth traveling dead end or (preferably) through a recycle loop, is fed to the pervaporation system 12 to maintain the fermentation product below the selected concentration for duration T2. Feedstock is added continuously or intentionally during T2. In the next step, fermentation and pervaporation are stopped and the broth is sent to the distillation column 14 for duration T3. In the next step, the equipment is cleaned or disinfected for duration T4. The process can then start over.

T2 is chosen considering a desire to minimize the relative duration of lost production time in T0 and T4 and a desire to keep the fermentator 18 working at a high rate of productivity. The rate of alcohol production declines over time within T2 as non-product inhibitory compounds collect in the fermentor 18. For example, for ethanol produced from sugar cane, T2 may start with a production rate of 50-100 g/L/h and decline to about one tenth of those values at the end of T2. Therefore the desire to extend T2 to minimize the relative duration of T0 and T4 must be balanced against the increased rate of production achievable by starting a new batch, among other factors and considerations. Using ethanol production from corn as an example, a fermentation batch without any product removed from the broth lasts about 1-2 days whereas T2 may be 4 days or more.

With a semi-continuous or extended batch process, feed to the pervaporation system 12 and distillation column 14 are discontinuous. To provide a more nearly constant feed stream to the pervaporation system 12 and distillation column 14, each may be connected to a plurality of fermentors 18 and service the fermentors 18 on a rotation basis. The number of fermentators 18 associated with a pervaporation system 12 may be different than the number of fermentators 18 associated with a distillation column 14. A holding tank may be used between one or more fermentors 18 and a distillation column 14 so that the fermentators 18 may be emptied rapidly while the distillation column receives a longer duration feed. Similarly, product from the distillation column 14 or pervaporation system 12 or both may be fed to a holding tank upstream of the vapour-permeation dewatering system 16.

The pervaporation system 12 includes modules of alcohol extraction pervaporation membranes and their associated equipment such as tanks or housings and pumps. The key requirements for the membrane modules are large surface area per unit footprint, tolerance to suspended solids and low cost per unit area. Ability to minimize liquid film and permeate side mass transfer resistances are also important. PV membrane modules are available from, for example, Applied Membrane Technology, Membrane Technology and Research, Pervatech BV and CeraMem Corp. The preferred configuration for the pervaporation system 12 is a shell-less module immersed in fermentor broth, or a tank receiving recycled fermentor broth, at ambient pressure. Pumps may be used to circulate broth from the fermentor 18, through the tank and back to the fermentor 18.

The membrane module configuration can be hollow fibre or flat sheet panel. Hollow fiber membranes can be arranged in modules of various configurations, for example as described in U.S. Pat. Nos. 6,325,928; 6,682,652; and 6,555,005, all of which are incorporated herein in their entirety by this reference to them. A flat panel configuration is preferred, however, because of reduced pressure losses on the permeate side which is of importance when operating under deep vacuum on the permeate side. Flat panel sheets with 2, 3 or 4 mm spacers produce low vacuum-side pressure losses and do not pose mechanical problems for assembly. The spacer is covered on both sides with two large (for example 1 to 4 square metres in area) membrane sheets and assembled typically with a frame to provide a panel. Several of the panels are stacked side by side with gaps of 5-15 mm to form modules which may in turn be assembled into cassettes that are arranged in covered open tanks. Methods of making flat plate membrane devices are described in U.S. Pat. Nos. 6,287,467; 5,482,625, 7,279,215; and 6,979,404, all of which are incorporated herein in their entirety by this reference to them.

In a flat panel module or cassette, permeate may be collected from or near one or both sides of the panels while a central part of the module is left open to vertical broth circulation. A biogas (mostly CO₂) is collected in the head space at the top of the tanks and is re-injected using a blower through a gas bubbling devices at the bottom of the membranes to evenly distribute the broth at the surface of the membrane, and reduce concentration and temperature polarization. A gas collection and recirculation system is shown, for example, in US publication number 2002-0170863 A1 which is incorporated herein in its entirety by this reference to it. Gas dispersion can be intermittent.

The membranes themselves need to be strong enough to not break, which would compromise the deep vacuum required for pervaporation. The membrane material may be coated on or impregnated into a non-woven substrate. Alternately, the pervaporation membrane material may be coated on, or fill the pores of, a MF or UF membrane, which may in turn be supported on a substrate. Coating a porous support with silicone rubber is described in U.S. Pat. No. 4,990,255 which is incorporated herein in its entirety by this reference to it. A cellulose acetate membrane coated with PDMS is described in Composite PDMS membrane with high flux for the separation of organics form water by pervaporation (Li, 2004). Li 2004 also gives various references describing coating silicone rubber on PES, PEI, PI, PAN, PE and ceramic supports. The separation membrane layer can be, for example, silicone rubber, without or with zeolite particles (e.g., silicalite-1 or 2) added to from a mixed matrix membrane.

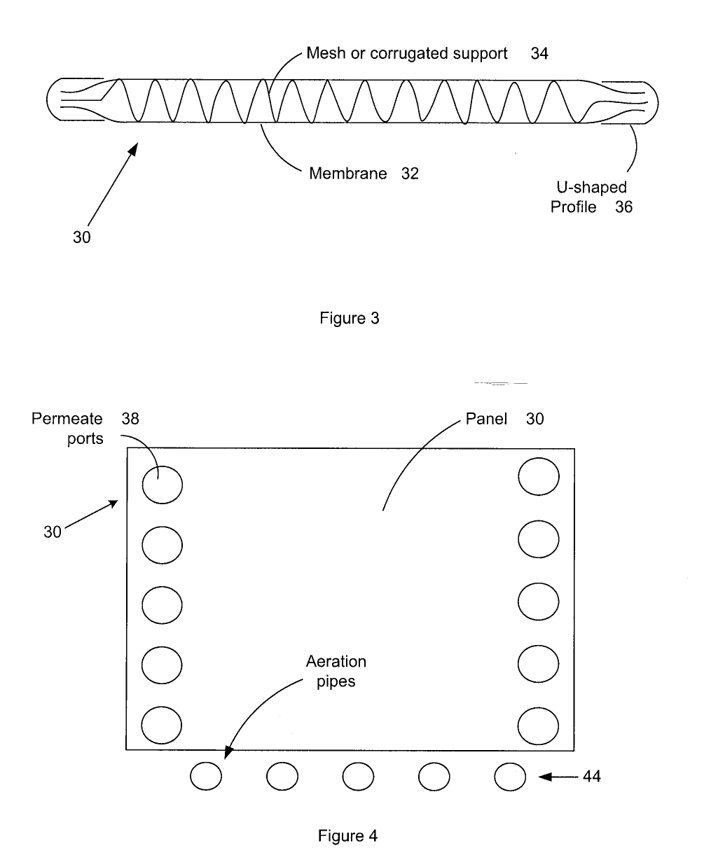

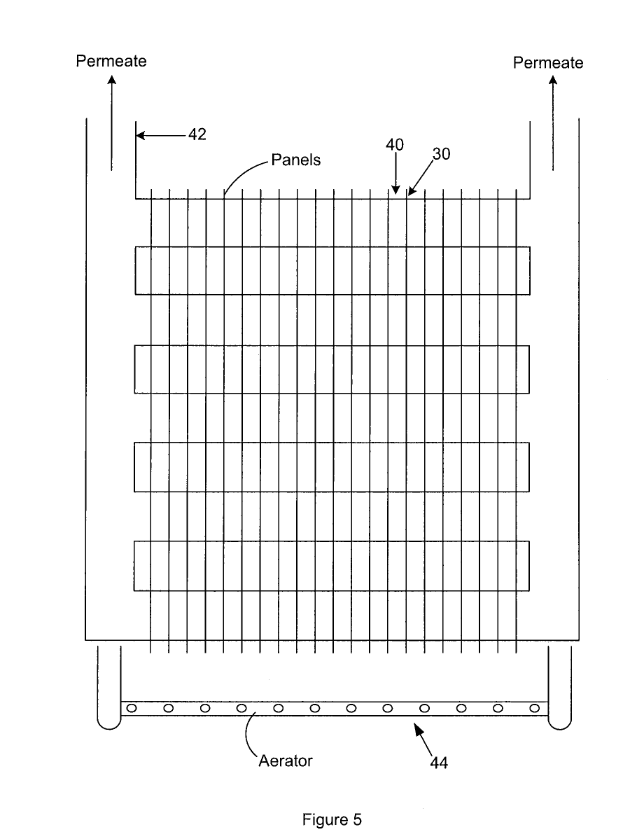

FIG. 3 shows the cross-section of a membrane element 30. A membrane sheet 32 is bonded to each side of a support 34, which may be for example a mesh or corrugated material. The four edges of the element 30 are flattened out and bonded and sealed together with an adhesive. The edges are protected with a U-shaped extrusion profile. The elements 30 are self-supporting but not rigid and about 3-4 mm thick. Referring to FIG. 4, permeate ports 38 are provided in multiple locations on the element 30, the number chosen to avoid excessive permeate side pressure drop. Referring to FIG. 5, multiple panels 30 are stocked side by side to form a cassette. The permeate ports 38 form permeate headers 40 which are in turn connected to one or more larger permeate collection pipes 42. Aeration pipes 44 are located below the elements 30.

The feed to the ethanol-extraction PV system 12 may be the broth as it resides in the fermentor 18, i.e. without substantial solids removal or heating. Some pre-screening may be done to remove solid particles to the extent required to protect the PV membrane. Any removed solids may be removed from the plant 10. Some heating may be required to compensate for the heat of evaporation through the PV membrane, but the temperature may be kept below the tolerance limit of the microorganisms, for example about 50 C. Preferably feed to the PV system 12 is kept at about the fermentation temperature, for example about 28-35° C. The PV membrane system 12 is preferably operated at low recovery (fraction of alcohol removed in a single pass through the PV system), for example less than <50% and preferably <20%. Higher recovery rates increase the amount of water in the permeate and increase energy usage per unit of product removed. While more selective membranes may permit a higher recovery before energy costs become prohibitive, low temperature and recovery operation lowers plant 10 energy costs, although requiring a greater membrane surface area to account for the reduced flux provided by operating at temperatures below the tolerance limit of the microorganisms.

Two options for handling permeate from the ethanol-extraction PV system are illustrated in FIG. 2. The preferred option for low selectivity membranes (FIG. 2a) involves condensation (with heat recovery) and re-boiling. This option works well with currently available membranes for example of silicone rubber with or without zeolite particles that have a selectivity of 8-15. For example, starting from a broth of 2-5 wt % ethanol, these membranes will produce permeate with an ethanol concentration between 20-50 wt %. Selectivity of silicone to butanol is a few times greater, but the concentration of butanol in the broth will be lower and so permeate butanol or ABE concentrations are similar. By re-boiling this condensate, a vapor enriched in ethanol in the 60-75 wt % range will be obtained. This vapor is suitable for use as a direct feed to a vapour permeation dewatering process described below. A potential additional benefit of this approach (i.e., using a membrane with limited selectivity) is that the permeate vapour still contains a significant amount of water that acts as a sweep gas for alcohol and reduces the requirement for a low vacuum pressure.

The second option for permeate handling (FIG. 2b) involves mechanically recompressing the vaporous permeate (with heating to avoid condensation) and feeding the vapor directly to the vapour permeation dewatering process. This option is preferred for membranes with selectivity greater than 15 for example mixed matrix or zeolite membranes, which would deliver permeate with an alcohol concentration of >60 wt %.

The distillation column 14 is operated continuously or semi-continuously to complement the operation of the ethanol extraction PV system 12. Its purpose is to process broth containing excess solids and non-product inhibitory compounds, and separate them into a solids-rich liquid phase at the bottom and an ethanol-rich vapor phase at the top. This distillation column 14 is similar to the equipment known as “beer column” in the ethanol industry. It produces a vaporous mixture at the top with an alcohol content between 50-70 wt %. In a plant 10 operated continuously, the distillation column 14 is used to control the average age of the yeast or bacteria cells in the fermentor, similar to what is done in the activated sludge process by controlling sludge age (the average residence time of bacteria in a biological wastewater treatment process). Yeast is renewed, on average, before budding about 15 times after which time they become generally inactive. Under semi-continuous operation of the plant 10, the distillation column 14 may start to be used with the start of a batch in the fermentor or after the yeast age or solids content has increased somewhat. Preferably, the distillation column 14 is used only after a batch is complete. In that case, the broth remaining in the fermentor 18 at the end of a batch is drained to the distillation column 14 to empty the fermentor 18 for the start of a new batch.

The vapour-permeation alcohol dewatering system 14 may be as sold by Vaperma using its Siftek™ membranes. These membranes and dewatering system are described in U.S. patent application Ser. Nos. 11/332,393 published as US 2006/0117955 A1 and 12/038,284 published as US 2008/0207959 A1, both of which are incorporated herein in their entirety by this reference to them. This system takes as a feed the streams from the pervaporation system 12 and the distillation column 14 to produce “dry” alcohol with water content normally smaller than 1 wt % but depending on final use and jurisdiction. The feed streams from the pervaporation system 12 and the distillation column 14 may be blended and fed to the vapor permeation system 16 simultaneously, or they may be fed to the vapor permeation system 16 sequentially, or by a combination of both methods.

The vapour permeation dewatering system 16 receives its feed from the two other systems in proportions x (from the distillation column 14) and (1-x) (from the pervaporation system 12). In general, it is desirable to minimize x because the distillation column consumes more energy than the PV system 12 to produce the same alcohol concentration. However, a desired minimum value for x exists for each fermentation system and feedstock, and is a function of benefits obtained by removing solids and non-product inhibitory compounds from the fermentor. The desire to remove suspended solids may be related to the PV membrane tolerance to solids while the desire to remove non-product inhibitory compounds is dictated by the fermentation process.

The plant 10 minimizes internal alcohol recycling and waste. The only side streams, the distillation column 14 bottom and the VP system 16 recycle stream, contain very little alcohol. These side streams may be treated and recycled within the plant 10.

Various examples for continuous fuel-grade ethanol production (99.2 wt %) from a broth containing 3 wt % ethanol are described below. The examples were extracted from complete mass and energy balances and aim at illustrating the energy benefits of the systems and methods described above.

The pervaporation membrane used for the calculations is a zeolite-filled silicone rubber as described by: L. M. Vane, V. V. Namboodiri and T. C. Bowen in Hydrophobic zeolite-silicone rubber mixed membranes for ethanol-water separation: Effect of zeolite and silicone component selection on pervaporation performance, Journal of Membrane Science (2007). The membrane has an ethanol/water selectivity of 25.

The vapor permeation membrane used in the calculations is as described in U.S. patent application Ser. No. 11/332,393 to Cranford et al., filed on Jan. 17, 2006 and published as U.S. 2006/0117955 A1 on Jun. 8, 2006.

The vapor permeation process is as described in Ethanol Processing with Vapour Separation Membranes, U.S. patent application Ser. No. 12/038,284 published as U.S. 2008/0207959 A1.

All of the distillation simulations were done in CHEMCAD 5.6.3. Membrane simulations were done with a model made by Vaperma Inc.

### EXAMPLE 1

### Pervaporation—Vapor Permeation

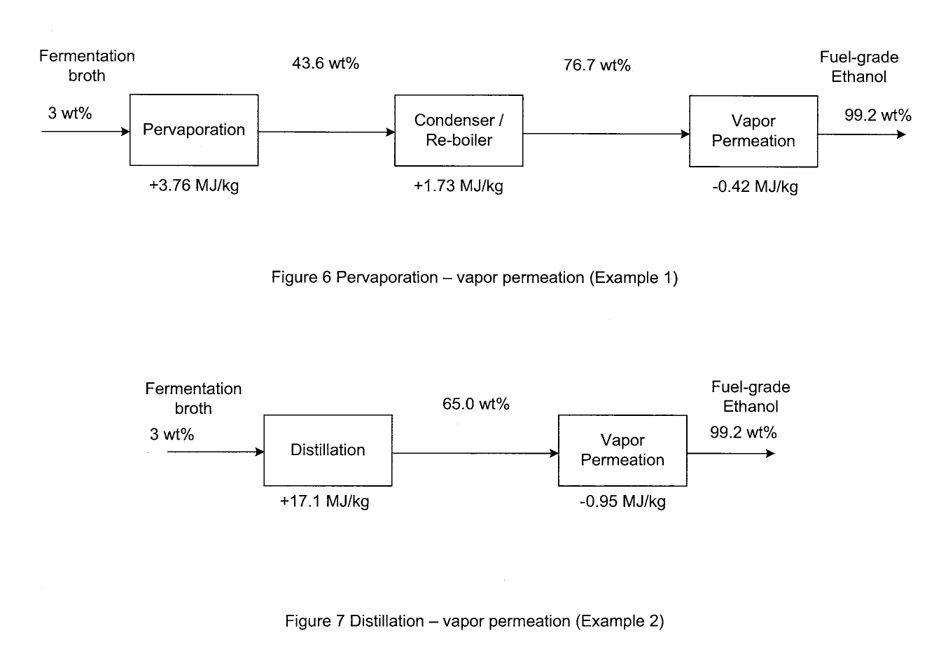

In this example, the full feed flow rate is processed by pervaporation and vapour permeation as illustrated in FIG. 6. The 3 wt % fermentation broth is feed into a pervaporation module which is operated at 20% recovery (i.e., 20% of the ethanol present in the feed is recovered through the pervaporation modules). No pre-treatment is provided (no suspended solids removal) but the feed is heated to compensate for heat of evaporation through the membrane. For a membrane with a selectivity of 25, the permeate contains 43.6% ethanol, as predicted by Equation 6 in Vane (2005). Heat must be added to the feed at a rate of 3.76 MJ/kg of ethanol to ensure that the broth returned to the fermentor is at the same temperature.

The pervaporation permeate is condensed and re-boiled. Since the pervaporation membranes are used at the temperature of the fermentor (28-35° C.), the heat of condensation is not recovered. The re-boiler is a simple distillation column with the following characteristics:
Trays=7
Reflux=0.5
% ethanol at the bottom=0.2%
Temperature=90° C.
Bottom pressure=200 kPa
Top pressure=170 kPa

This re-boiler brings the ethanol-water mixture to 76.7 wt % with a heat input of 1.73 MJ/kg ethanol.

The vapour at the top of the reboiler is super-heated to 105° C. and fed to a vapour permeation membrane where fuel grade ethanol is recovered as the retentate at 99.2 wt %. The water extracted on the permeate side under vacuum is condensed and the heat of condensation can be recovered since it is at a temperature of about 100° C. The heat recovered is equivalent to 0.42 MJ/kg ethanol produced. The mechanical recompression energy (e.g., vacuum pump) was neglected.

The net amount of energy to concentrate the ethanol from the broth at 3 wt % to fuel-grade at 99.2 wt % is therefore:

3.76+1.73−0.42=5.07 MJ/kg ethanol.

### COMPARATIVE EXAMPLE 2

### Distillation—Vapor Permeation

In this example, the full feed flow rate is processed by distillation and vapour permeation as illustrated in FIG. 4. The 3 wt % fermentation broth is feed into a distillation column with the following characteristics:
Trays=40
Reflux=5.26
% ethanol at the bottom=0.02%
Temperature=90° C.
Bottom pressure=200 kPa
Top pressure=170 kPa

This distillation column brings the ethanol-water mixture to 65.0 wt % with a heat input of 17.1 MJ/kg ethanol.

The vapour at the top of the reboiler is super-heated to 105° C. and fed to a vapour permeation membrane where fuel grade ethanol is recovered as the retentate at 99.2 wt %. The water extracted on the permeate side under vacuum is condensed and the heat of condensation can be recovered since it is at a temperature of about 100° C. The heat recovered is equivalent to 0.95 MJ/kg ethanol produced. The mechanical recompression energy (e.g., vacuum pump) was neglected.

The net amount of energy to concentrate the ethanol from the broth at 3 wt % to fuel-grade at 99.2 wt % is therefore:

17.1−0.95=16.15 MJ/kg ethanol.

### EXAMPLE 3

### Hybrid Process

In this example, the broth from the fermentor is split into two streams and processed via the pervaporation (1-x) or distillation (x) unit processes as illustrated in FIG. 1. The net energy to purify ethanol is dependent on the fraction “x” processed through distillation as shown in the table below. While the energy saved increases with decreasing “x”, it may be desirable to process a small fraction (x=10% or 25%) through distillation either while pervaporating, to purge excess suspended solids and reduce the build-up of inhibitory compounds in the fermentors, or near or after the end of a batch when increasing solid concentration might interfere with pervaporation. To increase solids flow to the distillation column 14, feed to the distillation column may be taken from the return line of the pervaporation system 12 recycle loop, or from the reject stream of a solid-liquid separation loop placed upstream of the pervaporation system 12.

| “x” | Net Energy | Energy |
|---|---|---|
| (% distillation) | MJ/kg ethanol | Saved (%) |
| 0 | 5.07 | 69 |
| 10 | 6.26 | 61 |
| 25 | 7.93 | 51 |
| 50 | 10.69 | 34 |
| 100 | 16.15 | 0 |

We claim:

1. A fermentation product production or dewatering plant comprising a fermentation product extraction pervaporation system and a vapour-permeation dewatering system.

2. The plant of claim 1 further comprising a distillation column.

3. The plant of claim 2 wherein the pervaporation system and distillation column are configured in parallel upstream of the vapour-permeation dewatering system.

4. A process of operating the plant of claim 1 comprising a step of removing a fermentation product from a fermentation broth through the pervaporation system followed by a step of removing water from the pervaporation product through the vapour-permeation dewatering system.

5. The process of claim 4 further comprising a step of removing alcohol from the broth through a distillation column.

6. The process of claim 4 wherein the pervaporation system is operated with feed broth at a temperature of 50 C less and fermentation product recovery of 50% or less.

7. The process of claim 4 wherein the pervaporation system is operated to maintain fermentation product concentration in a fermentor below a concentration at which product inhibition reduces the rate of product production by 30% or more.
